(19) United States
(12) Patent Application Publication
(10) Pub. No.: US 2026/0345923 A1
(43) Pub. Date: Nov. 5, 2026

(54) Injection Device with Needle Safety System

(71) Applicant: West Pharmaceutical Services, Inc., Exton, PA (US)

(72) Inventors: Nolan WALKER, Scottsdale, AZ (US); Randy GOLDFARB, Exton, PA (US); Rajkumar KARTHIKEYAN, Bengaluru (IN); Krishna JAYARAM, Bengaluru (IN)

(21) Appl. No.: 19/486,252

(22) PCT Filed: May 24, 2024
(86) PCT No.: PCT/US2024/031018

§ 371 Date: Nov. 20, 2025

Related U.S. Application Data
(60) Provisional application No. 63/504,269, filed on May 25, 2023.

(51) Int. Cl. A61M 5/50; A61M 5/142; A61M 5/158

(52) U.S. Cl. CPC A61M 5/50; A61M 2005/14252; A61M 2005/1426; A61M 2005/1585

(57) ABSTRACT

An injection device can include a housing having a contact surface to contact the injection site. The device can include an ortho-planar spring configured to be moved between a resting configuration and a compressed configuration. A needle hub with a needle is attached to the ortho-planar spring. A needle guard can be moved between an extended configuration, in which the needle guard covers the needle tip, and a retracted configuration, in which the needle guard does not cover the needle tip. A retaining clip can prevent movement of the needle guard from the extended configuration to the retracted configuration. The ortho-planar spring can be compressed when the housing is placed on the injection site. The retaining clip can be deflected away from the needle guard to allow relative movement between the needle guard and the needle hub so that the needle guard is retracted.

### CROSS REFERENCE TO RELATED APPLICATIONS

This application claims the benefit of U.S. Provisional Application No. 63/504,269, filed May 25, 2023, the entirety of which is incorporated herein for any and all purposes.

### TECHNICAL FIELD

This disclosure generally relates to devices and methods of injecting medicament into the user's skin, and more particularly relates to safety systems for preventing needle injuries during use of injection devices.

### BACKGROUND

During some medical treatments, medication cannot be administered to the patient via one or more discrete injections via traditional intradermal, intramuscular, or subcutaneous syringe injectors, and instead has to be administered to the patient over an extended period of time. Sometimes, such medications can be administered via an intravenous (IV) drip arrangement, where the medication is introduced into an IV bag and an IV tube is connected to the patient's blood vessel. Such administration can take many minutes or even hours, and the patient is limited in mobility due to the cumbersome IV setup. The patient also must get such treatment at a suitable medical facility. Wearable on-body injector devices have been developed that can be placed on the patient's skin and that can deliver a preset volume of medication at a predetermined delivery rate, thus allowing the patient to receive such treatment without the need for an IV setup as described above. However, there are still difficulties in administering larger volumes (greater than 1 mL) of medicament via such wearable injectors and administering to desired injection depths. Improper use or poor attachment of the device to the patient's skin can result in a “wet” injection, where some or all of the medicament can miss the intended delivery area, thus underdosing the patient. As such, there is a need for an improved wearable delivery device that can establish and maintain the desired contact with the surface of the patient's skin for the entire duration of the injection process.

### SUMMARY

The foregoing needs are met by various aspects of injection devices and injection safety systems disclosed. According to an aspect of this disclosure, an injection device for delivering a medicament to an injection site can include a housing configured to be placed on the injection site. The housing can define a contact surface thereon configured to contact the injection site. The device can further include an ortho-planar spring disposed in the housing and configured to be moved between a resting configuration and a compressed configuration, a needle hub fixedly attached to the ortho-planar spring, a needle affixed to the needle hub and having a needle tip at one end thereof, a needle guard having an extended configuration, in which the needle guard covers the needle tip, and a retracted configuration, in which the needle guard does not cover the needle tip, and a retaining clip configured to contact the needle guard and to prevent axial movement of the needle guard from the extended configuration to the retracted configuration. The ortho-planar spring can be configured to be moved to the compressed configuration when the housing is placed on the injection site such that the needle hub and the needle guard are moved axially toward the housing. The retaining clip can be configured to be deflected away from the needle guard to allow relative movement between the needle guard and the needle hub such that the needle guard is transitioned to the retracted configuration.

In some aspects, the ortho-planar spring may include a base affixed to the housing, a platform configured to be moved axially along an injection axis, and a plurality of flexible arms connecting the base to the platform, wherein the needle hub is fixedly attached to the platform.

Optionally, when the ortho-planar spring is in the resting configuration, the platform may be axially spaced away from the base along the injection axis.

When the injection device is placed on the injection site, the platform of the ortho-planar spring may be moved axially toward the base to place the ortho-planar spring into the compressed configuration, the ortho-planar spring being biased to its resting configuration.

Optionally, the injection device may include a spring configured to bias the needle guard to the extended configuration.

Optionally, the injection device may include an actuation hub that is axially movable along the injection axis and is configured to contact the retaining clip to cause the retaining clip to deflect away from the needle guard.

Optionally, the injection device may include a locking mechanism configured to prevent reuse of the injection device. The locking mechanism can include a protrusion on one of the actuation hub or the needle hub and a guide track on the other of the actuation hub or the needle hub. The protrusion can be configured to follow the guide track from a first position to a second position and be precluded from following the guide track from the second position to the first position. In some aspects, the protrusion may be disposed on the actuation hub, and the guide track may be defined on the needle hub. In some aspects, the guide track may be curved, and at least one of the actuation hub or the needle hub may be configured to rotate about the injection axis as the protrusion follows the curved guide track.

Optionally, the injection device may include a button disposed on the housing and configured to be depressed to cause the retaining clip to be deflected away from the needle guard.

Optionally, the contact surface of the housing can include an adhesive.

In some aspects, the needle may be a microneedle.

Optionally, the injection device may include a plurality of needles and/or a plurality of retaining clips.

Optionally, the injection device may include a medicament source configured to retain the medicament therein, the medicament source being in fluid communication with the needle. The medicament source may be disposed on or in the housing or, alternatively, may be spaced from the housing and connected to the housing by a tube.

In some aspects, the needle guard may be axially fixed relative to the needle hub when the needle guard is in the extended configuration.

In some aspects, the injection device can include a medicament therein or thereon. The medicament can be held in a medicament receptacle (e.g., a cartridge or syringe). The injection device may include an actuation mechanism configured to move the medicament through the injection device to a user.

In some aspects, the injection device may be a wearable injection device configured to be attached to the user. The injection device may be temporarily affixed to the user's body, for example, via an adhesive. In some aspects, the injection device may be surgically attached to the user's body, for example, via an implantable port.

In some aspects, the injection device may be a hand-held injector. The hand-held injector may be a self injector, an auto injector, or the like.

According to another aspect of this disclosure, a needle safety system for use with an injection device can include an ortho-planar spring having a base and a platform separated from the base by a plurality of flexible arms; a needle hub attached to the platform and including a needle thereon, the needle having a needle tip at one end thereof, a needle guard having an extended configuration, in which the needle guard covers the needle tip, and a retracted configuration, in which the needle guard does not cover the needle tip; and a retaining clip extending from the needle hub and configured to contact the needle guard, the retaining clip having an undeflected configuration and a deflected configuration. When the retaining clip is in the undeflected configuration, the retaining clip can contact the needle guard and prevent relative axial movement between the needle guard and the needle hub. When the retaining clip is in the deflected configuration, relative movement between the needle guard and the needle hub may be permitted.

According to another aspect of this disclosure, a method of delivery a medicament to an injection site using an injection device is described. The method can include the steps of positioning the injection device adjacent to the injection site to contact the injection site with the needle guard; contacting the contact surface of the housing with the injection site to cause the ortho-planar spring to move from the undeflected configuration to the deflected configuration; deflecting the retaining clips away from the needle guard to allow relative axial movement between the needle guard and the needle hub and causing the ortho-planar spring to be moved toward the undeflected configuration, wherein movement of the ortho-planar spring also causes movement of the needle hub toward the injection site to cause the needle to pierce the injection site, and movement of the needle hub causes the needle guard to be transitioned to the retracted configuration; delivering the medicament through the needle to the injection site; and moving the housing away from the injection site, such that the ortho-planar spring is in the undeflected configuration and the needle guard is transitioned to the extended configuration.

The injection device used in this method can include any of the injection devices described throughout this application. In some aspects, the injection device used in this method can include a housing configured to be placed on the injection site, where the housing can define a contact surface thereon configured to contact the injection site; an ortho-planar spring disposed in the housing and configured to be moved between a resting configuration and a compressed configuration, a needle hub fixedly attached to the ortho-planar spring, a needle affixed to the needle hub and having a needle tip at one end thereof, a needle guard having an extended configuration, in which the needle guard covers the needle tip, and a retracted configuration, in which the needle guard does not cover the needle tip, and a retaining clip configured to contact the needle guard and to prevent axial movement of the needle guard from the extended configuration to the retracted configuration. The ortho-planar spring can be configured to be moved to the compressed configuration when the housing is placed on the injection site such that the needle hub and the needle guard are moved axially toward the housing. The retaining clip can be configured to be deflected away from the needle guard to allow relative movement between the needle guard and the needle hub such that the needle guard is transitioned to the retracted configuration.

Optionally, the method can include a step of depressing a button configured to cause deflection of the retaining clips.

In some aspects, when the housing is moved away from the injection site, the ortho-planar spring is in the undeflected configuration, and the needle guard is in the extended configuration, the needle guard is precluded from again being transitioned to the retracted configuration.

In some aspects, the step of delivering the medicament occurs automatically after the needle pierces the injection site. Alternatively, the step of delivery the medicament can be actuated separately by the user of the injection device.

In yet another exemplary aspect, an injection device for delivering a medicament to an injection site may include a housing configured to be placed on the injection site. The housing can define a contact surface thereon configured to contact the injection site. The contact surface defines a base plane. The injection device further includes an ortho-planar spring disposed in the housing and being movable between a resting configuration and a compressed configuration. The injection device further includes a platform connected to the ortho-planar spring and configured to contact the injection site, the platform defining a needle plane extending therethrough. The injection device further includes a needle disposed on the platform of the ortho-planar spring and configured to be moved with the ortho-planar spring. When the ortho-planar spring is in the resting configuration, the platform can be positioned such that the needle plane is at a first angle relative to the base plane, and when the ortho-planar spring is in the compressed configuration, the platform can be positioned such that the needle plane is at a second angle relative to the base plane. The first angle can be different from the second angle.

Optionally, when the ortho-planar spring is in the resting configuration, the needle plane can be parallel to the base plane, and when the ortho-planar spring is in the compressed configuration, the needle plane can be non-parallel to the base plane.

Optionally, the ortho-planar spring can include a base and a flexible arm extending from the base to the platform, the flexible arm being deflectable relative to the base, such that when the flexible arm is deflected, the platform is moved relative to the base.

In some aspects, the ortho-planar spring can include two flexible arms, each flexible arm extending between the base and the platform. In some aspects, the ortho-planar spring can include three flexible arms.

In some aspects, the base plane can be non-parallel to the injection site, and the platform can be movable in a direction orthogonal to the injection site.

Optionally, the device can include a needle guard having an extended configuration, in which the needle guard covers the needle, and a retracted configuration, in which the needle guard does not cover the needle.

Optionally, the needle can be configured to be moved toward the injection site when the ortho-planar spring is moved into the compressed configuration.

### BRIEF DESCRIPTION OF THE DRAWINGS

The present application is further understood when read in conjunction with the appended drawings. For the purpose of illustrating the subject matter, exemplary aspects of the subject matter are shown in the drawings; however, the presently disclosed subject matter is not limited to the specific methods, devices, and systems disclosed. In the drawings:

FIG. 1 depicts an angled perspective view of an injection device according to an aspect of this disclosure;

FIG. 2 depicts another angled perspective view of the injection device of FIG. 1;

FIG. 3 depicts a cross-sectional view of the injection device of FIG. 1;

FIG. 4 depicts a cross-sectional view of a portion of an injection device according to an aspect of this disclosure;

FIG. 5 depicts a cross-sectional view of a portion of an injection device according to an aspect of this disclosure showing the needle guard in the extended configuration prior to use;

FIG. 6 depicts a cross-sectional view of the portion of an injection device of FIG. 5 showing engagement between the actuation hub and the retaining clips;

FIG. 7 depicts a cross-sectional view of the portion of an injection device of FIG. 5 showing the needle guard in the retracted configuration;

FIG. 8 depicts an angled perspective view of an ortho-planar spring according to an aspect of this disclosure;

FIG. 9 depicts another angled perspective view of the ortho-planar spring of FIG. 8;

FIG. 10 depicts a cross-sectional view of an injection device in one configuration according to an aspect of this disclosure;

FIG. 11 depicts a cross-sectional view of the injection device of FIG. 10 in another configuration according to an aspect of this disclosure;

FIG. 12 depicts a cross-sectional view of the injection device of FIG. 10 in yet another configuration according to an aspect of this disclosure;

FIG. 13 depicts a cross-sectional view of the injection device of FIG. 10 in yet another configuration according to an aspect of this disclosure;

FIG. 14 depicts a cross-sectional view of the injection device of FIG. 10 in yet another configuration according to an aspect of this disclosure;

FIG. 15A depicts a cross-sectional view of a locking mechanism for the injection device at a first position according to an aspect of this disclosure;

FIG. 15B depicts a cross-sectional view of the locking mechanism of FIG. 15A showing the locking mechanism at a second position according to an aspect of this disclosure;

FIG. 15C depicts a cross-sectional view of the locking mechanism of FIG. 15A showing the locking mechanism at a third position according to an aspect of this disclosure;

FIG. 16A depicts a cross-sectional view of a schematic of an injection device according to yet another aspect of this disclosure;

FIG. 16B depicts a cross-sectional view of a schematic of the injection device of FIG. 16A according to another aspect of this disclosure;

FIG. 17 depicts a flow chart showing a method of using an injection device according to an aspect of this disclosure; and

FIG. 18 depicts a top planar perspective view of an ortho-planar spring according to another aspect of this disclosure.

### DETAILED DESCRIPTION OF ILLUSTRATIVE EMBODIMENTS

Aspects of the disclosure will now be described in detail with reference to the drawings, wherein like reference numbers refer to like elements throughout, unless specified otherwise.

Certain terminology is used in the following description for convenience only and is not limiting. The words “proximal” and “distal” generally refer to positions or directions toward and away from, respectively, an individual using the mixing system. The words “axial”, “radial”, “above,” “below,” and similar terms designate directions in the drawings to which reference is made. The term “substantially” is intended to mean considerable in extent or largely but not necessarily wholly that which is specified. The terminology includes the above-listed words, derivatives thereof and words of similar import.

The term “plurality,” as used herein, means more than one. The singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

The transitional terms “comprising,” “consisting essentially of,” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristics” of the claimed invention. Embodiments described in terms of the phrase “comprising” (or its equivalents), also provide, as embodiments, those which are independently described in terms of “consisting of” and “consisting essentially of.”

Throughout this specification, words are to be afforded their normal meaning as would be understood by those skilled in the relevant art. However, so as to avoid misunderstanding, the meanings of certain terms will be specifically defined or clarified.

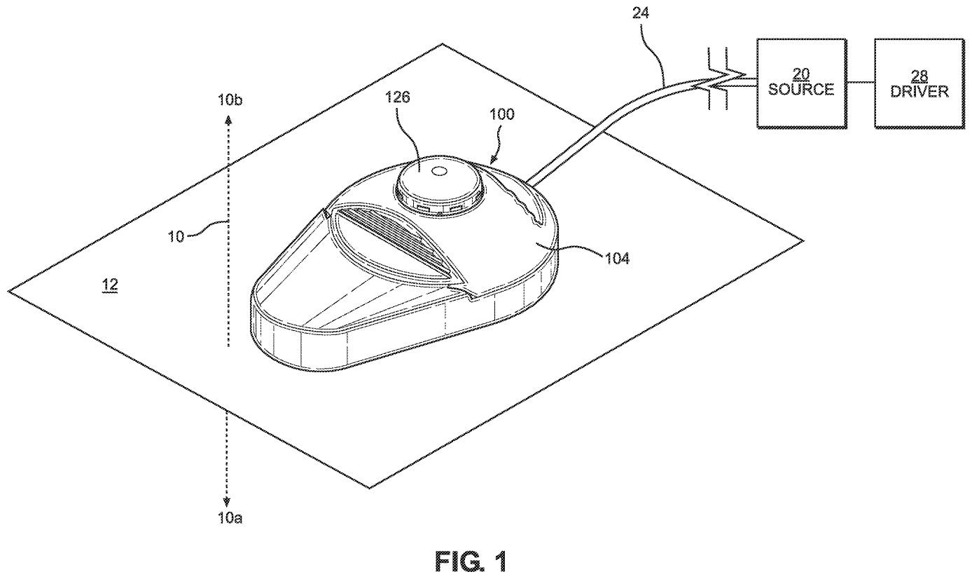

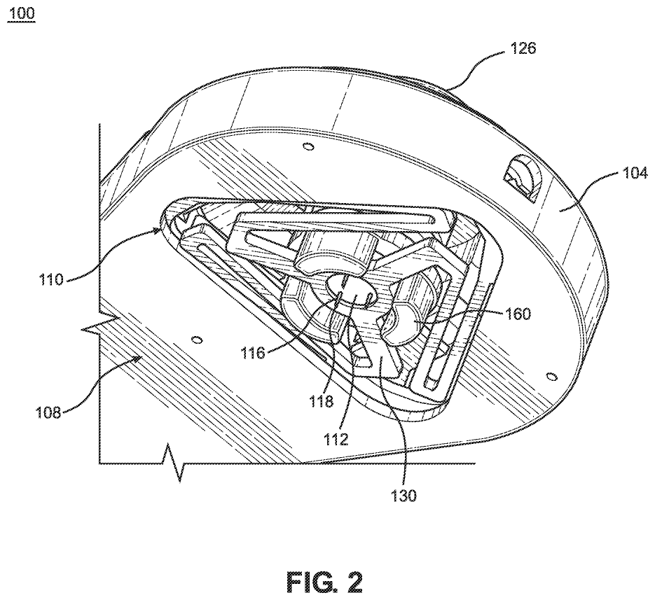

Disclosed are various aspects of injection devices that can be temporarily attached to a patient's skin to deliver a predetermined volume of medication to the patient. Referring to an exemplary aspect depicted in FIGS. 1 and 2, an injection device 100 can have a low-profile form factor and can include a housing 104 with a surface 108 that can contact the patient's skin. The injection device 100 receives one or more medicaments from a medicament source 20 via a line or tube 24 and into a needle 116. During use, the needle 116 pierces the patient's skin and forms a fluid connection between the medicament source 20 and the patient to allow the medicament to be delivered into the skin.

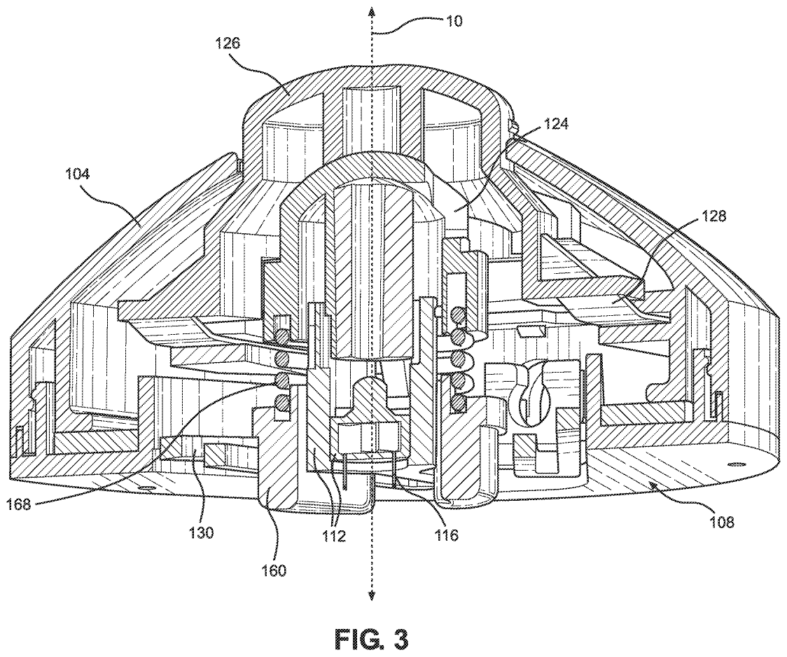

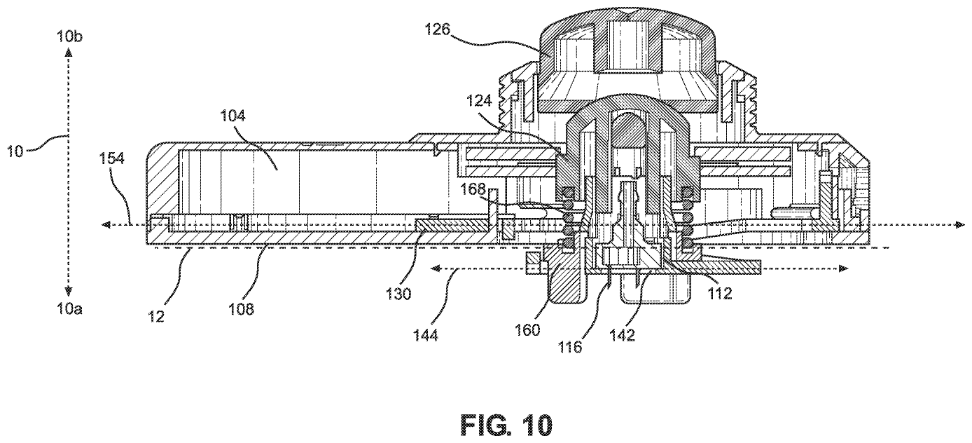

With reference to FIGS. 1-3, the housing 104 can include a surface 108 configured to contact the patient's skin at or near the desired injection position. The surface 108 may be substantially planar and may lie in, or define therethrough, a base plane 154. The injection site (e.g., the surface of the patient's skin) can define an injection plane 12. The base plane 154 can be parallel to the injection plane 12 at the surface 108 of the housing 104 (as can be seen in FIGS. 10-14). In some aspects, the surface 108 may include an adhesive thereon to facilitate temporary attachment of the injection device 100 to the skin. When the injection device 100 is on the patient's skin and the surface 108 is in contact with the skin, the injection plane 12 can be parallel to the base plane 154. As seen in FIG. 2, the surface 108 has an opening 110 therein through which the needle 116 may move. The opening connects the surface 108 with the interior of the housing 104. The needle 116 can be moved within the housing 104 and within and through the opening 110 during use. The needle 116 can move in a non-parallel direction relative to the surface 108. In some aspects, the needle 116 can be moved in a direction substantially perpendicular to the surface 108, for example, along an injection axis 10 (shown in FIGS. 1 and 3). The needle 116 can be moved in an injection direction 10a along the injection axis 10 or in a retraction direction 10b opposite the injection direction 10a. The base plane 154 can be substantially orthogonal to the injection axis 10.

The needle 116 defines a needle tip 118 at one end thereof. As the needle 116 is moved along the injection axis 10, the needle tip 118 can be positioned inside the housing 104, outside the housing 104 and spaced away from the surface 108, and at any position in-between. The needle 116 can be any suitable injection needle, such as a standard hypodermic needle or a microneedle. As used herein, the term “microneedle” is intended to mean a needle generally in the range of about 0.1 to about 3 mm in length or height and having an internal diameter of about 0.05 mm or less. The needle 116 can be hollow and define a channel therethrough. One or more medications from the medicament source 20 can be moved into and through the needle 116 and out of the needle 116 at the needle tip 118 during use. The needle 116 can be connected directly to a medicament source 20 in the injection device 100, for example inside the housing 104, or, alternatively, the needle 116 can be connected via a line or tube 24 to a medicament source 20 that is spaced from the housing 104. In some aspects, the injection device 100 can include a plurality of needles 116. The plurality of needles 116 may all be the same types and dimensions, or, alternatively, they can be different types and/or different dimensions.

The medicament can be moved from the source 20 to the injection device 100 by a drive system 28. The drive system 28 can be external to the injection device 100 as shown in FIG. 1, or, alternatively, can be part of the injection device 100. The drive system 28 can include any suitable mechanism for driving flow of a liquid, such as peristaltic pumps, gear pumps, piston pumps, or another suitable pumping mechanism.

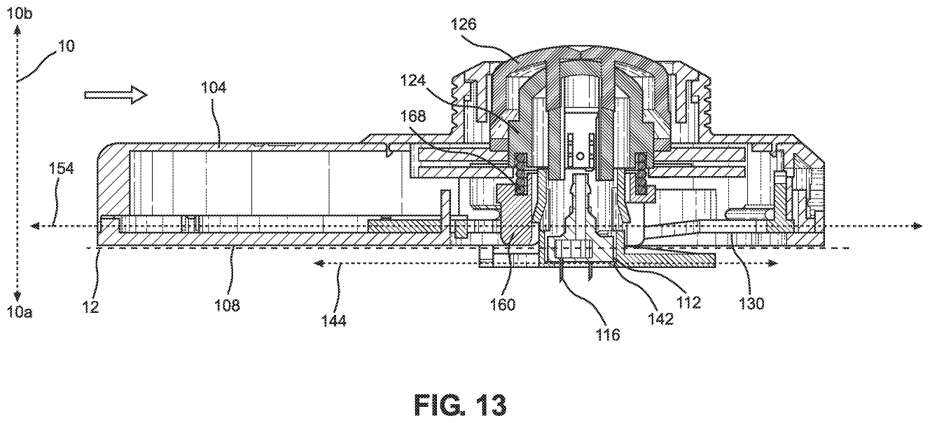

The injection device 100 includes a needle guard 160. The needle guard 160 can be positioned coaxially to the needle 116 (or to the plurality of needles 116). The needle guard 160 can be axially movable along the injection axis 10 relative to the housing 104 and/or to the needle 116 during use. The needle guard 160 can be positioned in a retracted configuration (see, e.g., FIG. 13) or an extended configuration (see, e.g., FIGS. 10-12) along the injection axis 10. When the needle guard 160 is in the retracted configuration, the needle tip 118 is axially spaced away from the needle guard 160 along the injection axis 10. In the retracted configuration, the needle guard 160 does not cover the needle tip 118, and the needle tip 118 is accessible and can contact and pierce the skin. When the needle guard 160 is in the extended configuration, the needle guard 160 extends over the needle tip 118, such that the needle tip 118 is not axially spaced from the needle guard 160 along the injection axis 10. In the extended configuration, the needle guard 160 obstructs access to the needle tip 118, thus decreasing risk of needle stick injury by the user. The needle guard 160 can be moved between the retracted and extended configurations along the injection axis 10. The needle guard 160 can be biased to the extended configuration via a biasing element. In some aspects, the biasing element can include a coil spring 168. The coil spring 168 can be compressed when the needle guard 160 is in the retracted configuration and can be less compressed (or relaxed) when the needle guard 160 is in the extended configuration.

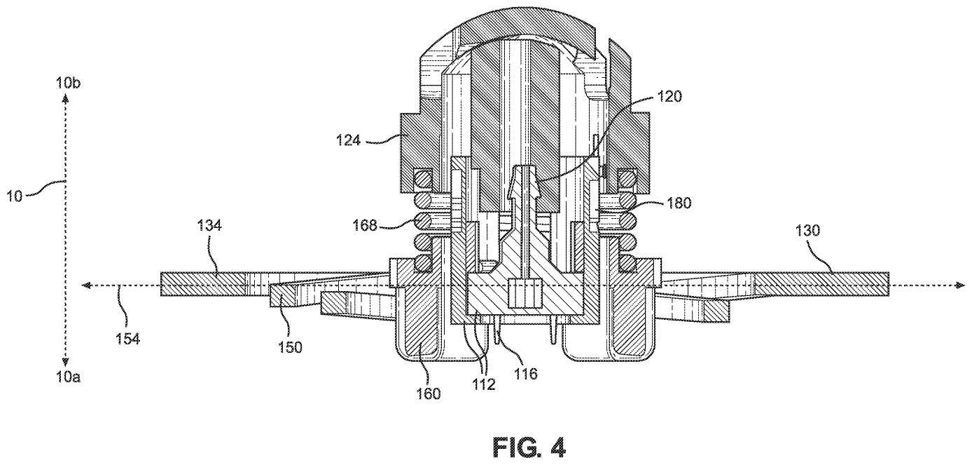

With further reference to FIGS. 4-7, the needle 116 can be attached to, affixed to, or otherwise secured to, a needle hub 112. The needle hub 112 can be moved in both opposing directions (the injection direction 10a and the retraction direction 10b) along the injection axis 10. Movement of the needle hub 112 can cause movement of the attached needle 116. The needle guard 160 can be releasably connected to the needle hub 112. The needle guard 160 can be positioned in its extended configuration or its retracted configuration depending on movement of the needle hub 112. The needle guard 160 can extend partially or fully around the circumference of the needle hub 112. In some aspects, the needle guard 160 can be disposed within the needle hub 112, such that the needle hub 112 extends partially or fully around the circumference of at least a portion of the needle guard 160. In some aspects, the needle hub 112 and/or the needle guard 160 can have a desired contour or shape designed to complement the expected shape of the surface of the skin where the injection device 100 is expected to be used. In some aspects, the needle hub 112 and/or the needle guard 160 can include an adhesive configured to releasably retain the needle hub 112 and/or the needle guard 160, respectively, on the surface of the skin.

The needle hub 112 can include one or more retaining clips 114 configured to prevent relative movement between the needle guard 160 and the needle hub 112 such that the needle guard 160 is in the retracted configuration. The retaining clips 114 can be positioned circumferentially along the needle hub 112, for example, between the needle hub 112 and the needle guard 160. The needle hub 112 can include 1, 2, . . . , 8, or more retaining clips 114. In some aspects, each retaining clip 114 can extend at least partially around the circumference of the needle hub 112.

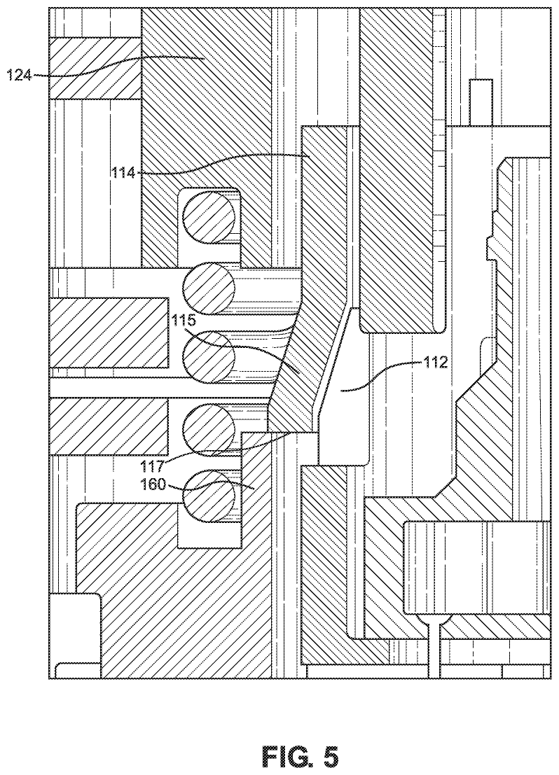

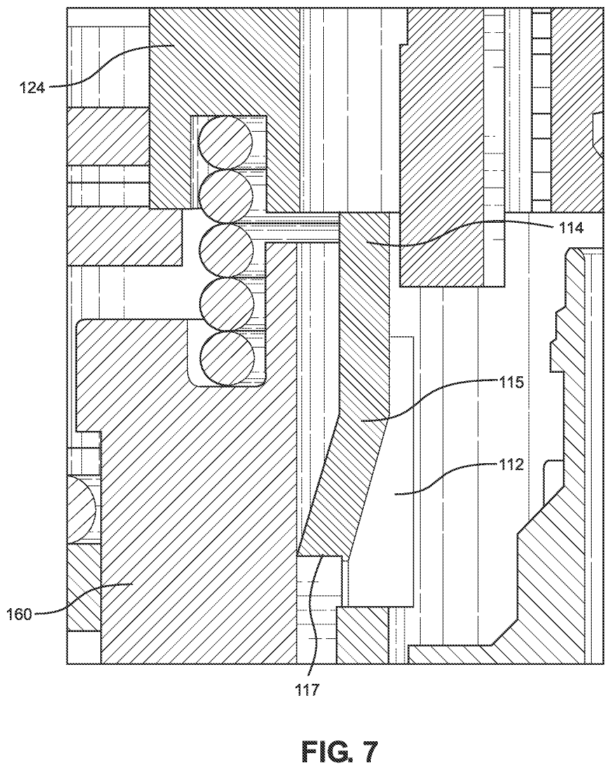

The retaining clips 114 can be deflectable, for example, in a radial direction toward the center of the needle hub 112. When the needle guard 160 is in the extended configuration, the retaining clips 114 contact the needle guard 160 and prevent relative axial movement between the needle guard 160 and the needle hub 112 (see, e.g., FIG. 5). When the retaining clips 114 are deflected inward toward the center of the needle hub 112, they no longer axially contact the needle guard 160, and relative movement between the needle guard 160 and the needle hub 112 is permitted (see, e.g., FIG. 7). This can cause the needle guard 160 to move from the extended configuration to the retracted configuration, thus exposing the needle tips 118. It will be understood that relative movement between the needle hub 112 and the needle guard 160 can include movement of the needle guard 160 relative to the needle hub 112, movement of the needle hub 112 relative to the needle guard 160, or movement of both the needle hub 112 and the needle guard 160.

As shown in FIGS. 5-7, the retaining clips 114 can include a ramp portion 115 that extends obliquely away from the needle hub 112 and from the injection axis 10. A distal end 117 of the ramp 115 is configured to contact the needle guard 160 when the needle guard 160 is in the extended configuration as shown in FIG. 5. Such contact blocks relative axial movement between the needle hub 112 and the needle guard 160 that would put the needle hub 112 into the retracted configuration and thus expose the needle tips 118.

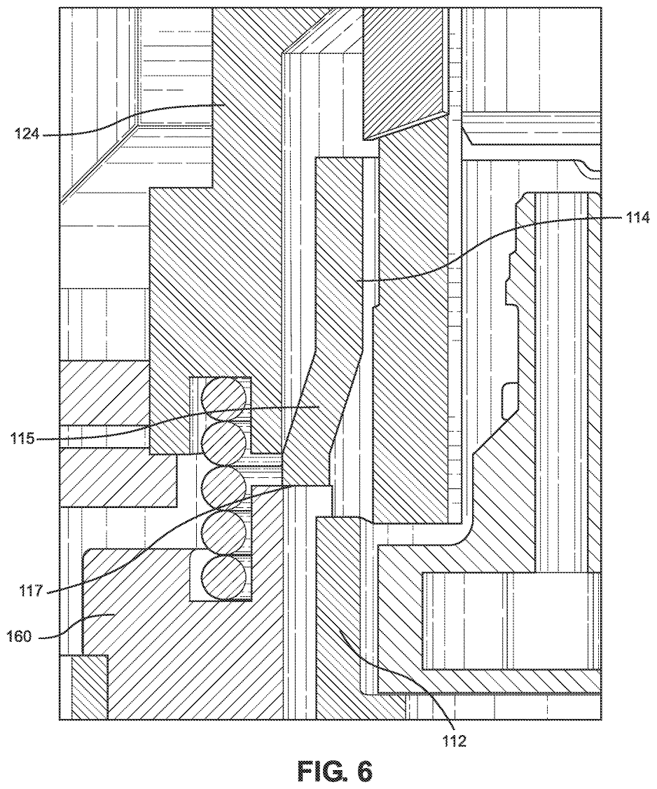

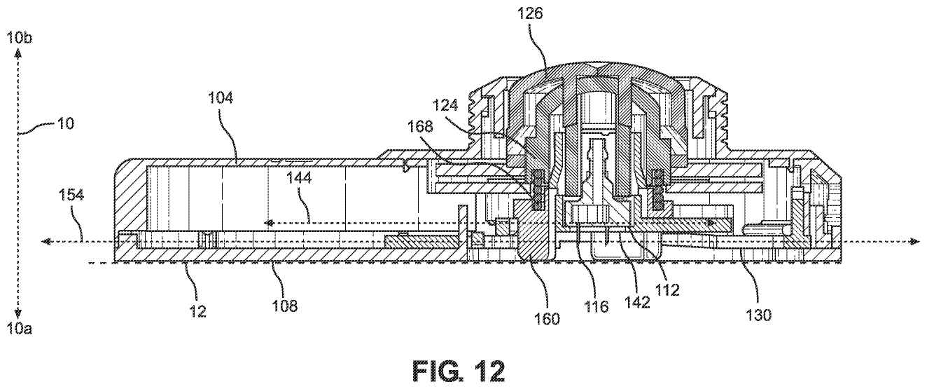

The retaining clips 114 can be deflected by an actuation hub 124 movable along the injection axis 10. The actuation hub 124 can be moved between a rest position (shown in FIGS. 5, 10, and 11) and an engaged position (shown in FIGS. 6 and 12). In the rest position, the actuation hub 124 is spaced axially away from the retaining clips 114 and does not contact the retaining clips 114. In the engaged position, the actuation hub 124 is axially moved to contact the retaining clips 114. The actuation hub 124 can contact the ramp portions 115 as it is moved axially toward the retaining clips 114. Continued movement of the actuation hub 124 toward the retaining clips 114 causes the actuation hub 124 to slide along the ramp portions 115. The force applied by the actuation hub 124 onto the ramp portions 115 causes the ramp portions 115 to deflect radially inward towards the needle hub 112. As each ramp portion 115 deflects radially inward, each distal end 117 of the retaining clip 114 is also moved inward. When the actuation hub 124 has traveled a predetermined distance, the retaining clips 114 are deflected inward such that the distal ends 117 are no longer in axial alignment with the needle guard 160 and thus no longer axially block relative movement between the needle guard 160 and the needle hub 112. At this point, the needle hub 112 can be moved and can move the needle tips 118 beyond the needle guard 160. Thus, the needle guard 160 is in its retracted configuration at this point. It should be understood that the transition between the extended and retracted configurations can be caused by movement of the needle hub 112, the needle guard 160, or both components.

When the needle guard 160 is in the retracted configuration as shown in FIG. 7, the retaining clips 114 can be in the deflected configuration and be biased radially outward away from the center of the needle hub 112 to their undeflected configurations. When the needle guard 160 and/or the needle hub 112 are again axially moved so as to place the needle guard 160 back into its extended configuration, the retaining clips 114 can return to their undeflected configurations. During such axial movement, the distal ends 117 of each retaining clip 114 are moved until they axially clear the needle guard 160. At that point, the bias in the retaining clips 114 can cause the ramp portions 115 to return to their undeflected configurations, thus moving the distal ends 117 back into axial alignment with the needle guard 160 as shown in FIG. 5. This can prevent further exposure of the needle tips 118 by returning the needle guard 160 back to its extended configuration.

Although the exemplary description above includes the needle guard 160 circumferentially surrounding the needle hub 112 and at least a portion of each retaining clip 114, it should be understood that a different relative arrangement of these components is also foreseen. For example, at least a portion of the needle guard 160 could be disposed inside the needle hub 112, such that the needle hub 112 surrounds the needle guard 160. In such embodiments, the retaining clips 114 can be arranged to deflect radially outward away from the center of the needle hub 112.

As explained above, the needle guard 160 can be fixedly attached to the needle hub 112 due, at least in part, to the engagement of the retaining clips 114 with the needle guard 160. During some stages of operation of the injection device 100, as will be further detailed below, the needle guard 160 can be moved axially, for example along the injection axis 10, along with the needle hub 112. That is, when the needle hub 112 is moved, the needle guard 160 can also be moved.

The needle hub 112 can be moved by one or more biasing members. In some aspects, the needle hub 112 can be moved in a first axial direction (for example in the injection direction 10a) along the injection axis 10 by one biasing member and in a second axial direction opposite the first axial direction (for example in the retraction direction 10b) by a second, separate biasing member. In other aspects, the needle hub 112 can be moved in both axial directions along the injection axis 10 by a single biasing member. Each biasing member can include a spring.

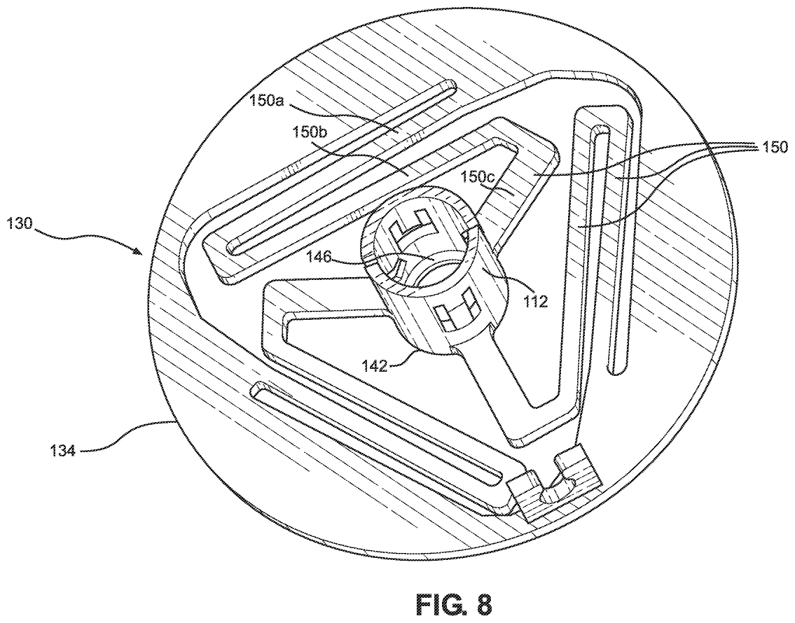

With continued reference to FIGS. 2-4 and with further reference to FIGS. 8-14, the injection device 100 can include a biasing member configured to move the needle hub 112 along the injection axis 10. The biasing member can include a spring, for example, an ortho-planar spring 130. The ortho-planar spring 130 can be configured to move the needle hub 112 in the injection direction 10a away from the injection device 100 and in the retraction direction 10b toward the injection device 100. For purposes of this disclosure, an ortho-planar spring can include a spring that can be either fabricated in, or compressed down into, a single plane, with motion out of that plane. One advantage of an ortho-planar spring over traditional coil springs is that an ortho-planar spring is more compact and can be easily manufactured.

The ortho-planar spring 130 can include a base 134 configured to be fixed to the housing 104 of the injection device 100. The base 134 can be designed to remain substantially fixed along the injection axis 10 relative to the housing 104. The base 134 can be secured to the housing 104 via any suitable mechanisms, such as fasteners, adhesive, friction fit, compression fit, snap fit, or be held in place by one or more other components. In some aspects, the base 134 can be integrally formed along with the housing 104. Alternatively, the base 134, and the rest of the ortho-planar spring 130, can be separate and distinct components from the housing 104. The base 134 can be any suitable shape, such as triangular, circular, oval, rectangular, pentagonal, hexagonal, octagonal, star-shaped, or another suitable shape. In the exemplary embodiments shown in the FIGs., the base 134 is circular, but this depiction is not intended to limit the shape of the base 134 or the ortho-planar spring 130.

The base 134 can be disposed within the housing 104 such that the ortho-planar spring 130 is positioned in the desired orientation relative to the rest of the injection device 100 and/or the injection surface (e.g., the patient's skin).

The ortho-planar spring 130 further includes a platform 142 configured to be movable relative to the base 134 toward and away from the base plane 154. The platform 142 can be configured to contact the injection site (e.g., the patient's skin) at a surface defined on the platform 142. The contact surface on the platform 142 can define a needle plane 144 (see FIG. 10). The needle plane 144 can be substantially parallel to the base plane 154 and/or the injection plane 12. In some aspects, the ortho-planar spring 130 can be moved relative to the housing 104 such that the needle plane 144 is not parallel to the base plane 154. In some aspects, during use, the needle plane 144 may be substantially parallel to the injection plane 12 but not parallel to the base plane 154 as will be described further below.

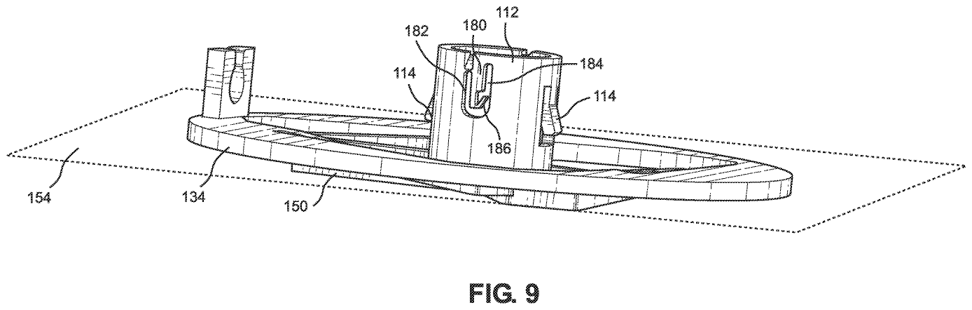

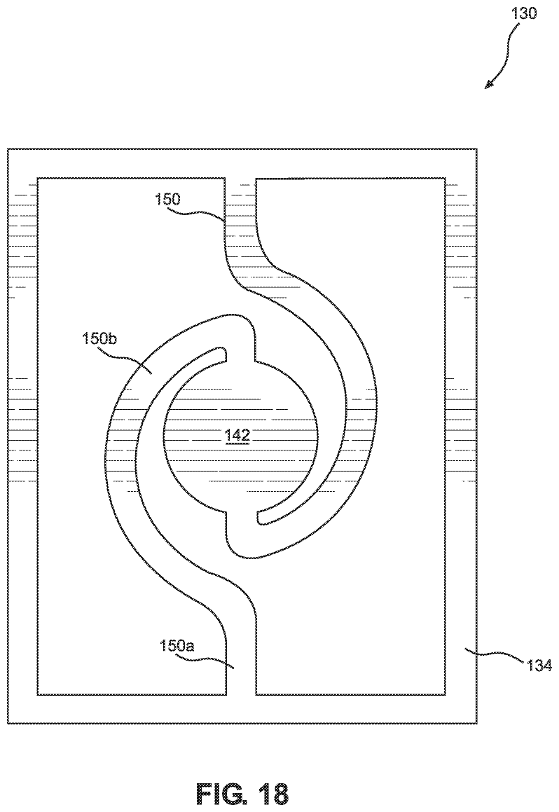

The platform 142 is attached to the base 134 via one or more flexible arms 150. The flexible arms 150 are configured to be deflected toward or away from the base plane 154. In some aspects, movement of the platform 142 can cause deflection of the flexible arms 150; in other aspects, deflection of the flexible arms 150 can cause movement of the platform 142. The flexible arms 154 can include any suitable shape, size, dimension, and elasticity based on the desired range of movement of the ortho-planar spring 130, and the exemplary embodiments shown herein are not intended to limit the structural design aspects of viable ortho-planar springs that can be used in the injection device 100. For example, the flexible arms 150 extending between the base 134 and the platform 142 can be straight, curved, or angled. The ortho-planar spring 130 can include one or a plurality of flexible arms 150, for example, 1, 2, . . . , 12, or another suitable number of flexible arms 150. In some aspects, the ortho-planar spring 130 can include at least three flexible arms 150 to improve stability of the ortho-planar spring 130 during use. In some particular non-limiting exemplary aspects, the ortho-planar spring 130 can include exactly three flexible arms 150 arranged radially equidistant from each other (see, e.g., FIGS. 8 and 9). In other non-limiting exemplary aspects, the ortho-planar spring 130 can include two flexible arms 150 arranged opposite each other (see, e.g., FIG. 18). Utilizing an ortho-planar spring 130 with fewer flexible arms 150 (for example, two flexible arms instead of three) allows for increased degrees of rotation and translation of the platform 142 and may be advantageous in applications where the injection surface (i.e., the patient's skin) is rough, not flat, irregularly shaped, or smaller than the device 100. The flexible arms 150 can be arranged in any suitable pattern to achieve the desired range of motion of the ortho-planar spring 130, such as a spiral, wheel-and-spoke, zig zag, or another geometric pattern. In some aspects, all of the flexible arms 150 are of the same size, shape, dimension, material, and elastic properties. In other aspects, some of the flexible arms 150 can differ from other flexible arms 150 in size, shape, dimension, material, and/or material or elastic properties. The flexible arms 150 can be uniform throughout between the base 134 and the platform 134, or, alternatively, each flexible arm 150 can have different physical and/or mechanical properties along different portions thereof between the base 134 and the platform 142.

In some aspects, each flexible arm 150 can include one or more segments extending between the base 134 and the platform 142. As shown in the exemplary aspect of FIG. 8, for example, the base 134 can be connected to the platform 142 by a flexible arm 150 having a first flexible arm segment 150a, a second flexible arm segment 150b, and a third flexible arm segment 150c arranged in series. The first flexible arm segment 150a can contact the base 134 at one end thereof and contact the second flexible arm segment 150b at the other end thereof. The second flexible arm segment 150b can contact the first flexible arm segment 150a at one end thereof and contact the third flexible arm segment 150c at the other end thereof. The third flexible arm segment 150c can contact the second flexible arm segment 150b at one end thereof and contact the platform 142 at its other end. Each of the first, second, and third flexible arm segments 150a, 150b, and 150c can have the same mechanical and physical parameters as any of the other flexible arm segments, or, alternatively, some of the flexible arm segments may differ in mechanical and physical characteristics from one or more other flexible arms 150 or flexible arm segments. In some aspects, all of the flexible arms 150 of the ortho-planar spring 130 can have the same number, arrangement, and parameters of flexible arm segments, or, alternatively, some of the flexible arms 150 within the ortho-planar spring 130 can have different arrangements thereof, for example having different flexible arm segments on different flexible arms. Although the exemplary embodiment described above includes three flexible arm segments 150a, 150b, and 150c arranged in series, it should be understood that any suitable number of flexible arm segments can be arranged on the flexible arm 150, including 1, 2, 3, 4, or another suitable number. For example, in the non-limiting exemplary aspect depicted in FIG. 18, each of the flexible arms 150 is shown having a first flexible arm segment 150a and a second flexible arm segment 150b connected in series between the base 134 and the platform 142. It will be appreciated that, in some aspects, a series of flexible arm segments (such as the first, second, and third flexible arms 150a, 150b, and 150c) could be collectively referred to as a single flexible arm 150 as described in the paragraphs above.

The flexible arms 150 serve the purpose of connecting the platform 142 to the base 134 and allowing movement of the platform 142 relative to the base 134. The flexible arms 150 can have a desired elasticity to allow for deflection away from or toward the base plane 154. The flexible arms 150 can be formed to be biased toward the base plane 154. That is, in their undeflected position at rest, the flexible arms 150 would be substantially parallel to the base 134 and base plane 154. In this position, the platform 142 would also be parallel to the base 134 and the base plane 154. The platform 142 can be moved away from the base plane 154 along the orthogonal injection axis 10 in either direction, thus deflecting the flexible arms 150. When the platform 142 is positioned away from the base plane 154, the flexible arms 150 can be biased towards their undeflected position. When the biasing force of the flexible arms 150 overcomes the force moving or keeping the platform 142 away from the base plane 154, the flexible arms 150 can return to their undeflected position, thus moving the platform 142 towards the base plane 154 as well.

In some aspects, the flexible arms 150 can be formed to be biased to a position along the injection axis 10 that is spaced from the base plane 154. In such aspects, when the flexible arms 150 are at their undeflected position at rest, at least parts of the flexible arms 150 would not be in the base plane 154 and would instead be spaced away from the base plane 154 axially along the injection axis 10. As such, the platform 142 would also be spaced away from the base plane 154. If the platform 142 is moved toward the base plane 154, the flexible arms 150 would deflect and would exert a biasing force against the platform 142 to return to their undeflected position. When the biasing force of the flexible arms 150 overcomes force responsible for moving the platform 142, the bias in the flexible arms 150 can cause the flexible arms 150 to return to their undeflected position, thus moving the platform 142 as well. As shown in the exemplary embodiment of FIGS. 10-14, in some aspects, the flexible arms 150 can be formed so as to extend away from the base plane 154 in the injection direction 10a, such that the platform 142 is axially spaced away from the base plane 154 in the injection direction 10a. The platform 142 can be moved axially in the retraction direction 10b, thus deflecting the flexible arms 150. When the flexible arms 150 are deflected in this embodiment, the flexible arms 150 are biased in the injection direction 10a and apply a biasing force to the platform 142 in the injection direction 10a. When one or more flexible arms 150 is deflected, the ortho-planar spring 130 can be in a compressed configuration. When none of the flexible arms 150 are deflected, the ortho-planar spring 130 can be in a resting configuration.

The platform 142 can include one or more components of the injection device 100 that can be moved when the platform 142 is moved. In some aspects, the needle hub 112 can be affixed to the platform 142. The needle hub 112 can be attached via any suitable attachment method, such as fasteners, adhesives, snap fit, friction fit, compression fit, or the like. In some aspects, the needle hub 112 can be formed integrally with the platform 142 and the rest of the ortho-planar spring 130. The needle guard 160 can be connected to the ortho-planar spring 130 via its engagement with the needle hub 112 as described above.

During use of the injection device 100, some of the components described above can be moved to initiate, perform, or complete injection of medicament into the patient. The needle guard 160 can be moved to its retracted position to expose the needle tip 118, the needle 116 can be moved towards and into the patient's skin at the injection site, and medicament can be delivered through the needle 116. The needle 116 can be retracted from the injection site, and the needle guard 160 can be moved to its extended configuration to cover the needle tip 118. Some or all of the events associated with using the injection device 100 can be controlled via a pre-programmed electronic device connected to, or housed within, the injection device 100, or some or all of these events can be controlled by manual actuation, for example, by the user of the injection device 100.

The injection device 100 can include an actuator configured to initiate one or more steps in the injection process. As shown in FIGS. 1-3, the injection device 100 can include a button 126 configured to be depressed by the user of the injection device 100. The button 126 can be on or in the housing 104 and can extend, at least in part, outside of the housing 104. The button 126 can be movable relative to the housing 104 and to one or more components inside the housing 104. In some aspects, the button 126 can be biased away from the housing 104 by a biasing member, such as a spring. As depicted in the exemplary embodiment of FIGS. 1-3, the spring could include a leaf spring 128, but it should be understood that another type of suitable spring could be used instead, for example a coil spring. To depress the button 126, the user can apply a force to the button 126 against the biasing force of the leaf spring 128 and that is greater than the biasing force; when the user's force is removed from the button 126, the biasing force of the leaf spring 128 can cause the button 126 to return to its undepressed position.

In some aspects, the button 126 can be depressed in the injection direction 10a. As the button 126 is moved in the injection direction 10a, the button 126 can contact the actuation hub 124 described above. Continued movement of the button 126 after contacting the actuation hub 124 can cause the actuation hub 124 to also move in the same injection direction 10a. When the actuation hub 124 travels a predetermined distance, the actuation hub 124 can engage with the retaining clips 114 and cause the retaining clips 114 to deflect and cause movement of the needle hub 160 as was previously described.

It should be appreciated that the mechanical button 126 described above could be an electronic button configured to receive input via capacitive, resistive, tactile, or other suitable mechanism from the user of the injection device 100. Actuation of the electronic button could cause a processor (not shown) within the injection device 100 to send electronic signals to an actuator, such as a motor (not shown), that could, in turn, cause movement of the actuation hub 124.

In some aspects, the button 126 could be integrally formed with the actuation hub 124, such that the actuation button 126 and the actuation hub 124 are a single component. In such aspects, the user could depress this component (as a combination of the button 126 and the actuation hub 124) to move the combination of the button 126 and the actuation hub 124 along the injection axis 10 simultaneously to cause deflection of the retaining clips 114.

Actuation of the button 126 can actuate the drive system 28 to cause the medicament to be moved from the medicament source 20 to the needle 116. In some aspects, actuation of the drive system 28 can be caused by another actuation component separate from the button 126.

The injection device 100 can be designed for different uses. In some aspects, the injection device 100 can be used multiple times for multiple injections. In such cases, the user can actuate the injection device 100 multiple times to cause medicament to be delivered as described throughout this application. The injection device 100 can be reused with the same medicament source 20 or with another medicament source 20.

In some aspects, it may be advantageous to limit or prevent reuse of the injection device 100 to reduce the risk of needlestick injury, medicament contamination, improper dosage, or other common problems associated with self-delivery of medications.

The injection device 100 can include a mechanism for restricting reuse so that a user cannot actuate delivery of medicament a second or subsequent time after a first actuation of the delivery device 100.

In some aspects, the delivery device 100 can prevent reuse thereof by include a mechanism that prevents the needle guard 160 from being moved from its extended configuration to its retracted configuration. If the needle guard 160 cannot be moved to its retracted configuration, the needle tip 118 cannot be exposed, and thus, the needle 116 cannot be injected into the user's skin. The delivery device 100 can permit movement of the needle guard 160 from its extended configuration to its retracted configuration a single time, and then movement from its retracted configuration to its extended configuration after the injection process is complete. Subsequent repeated movement of the needle guard 160 to its retracted configuration may be prevented.

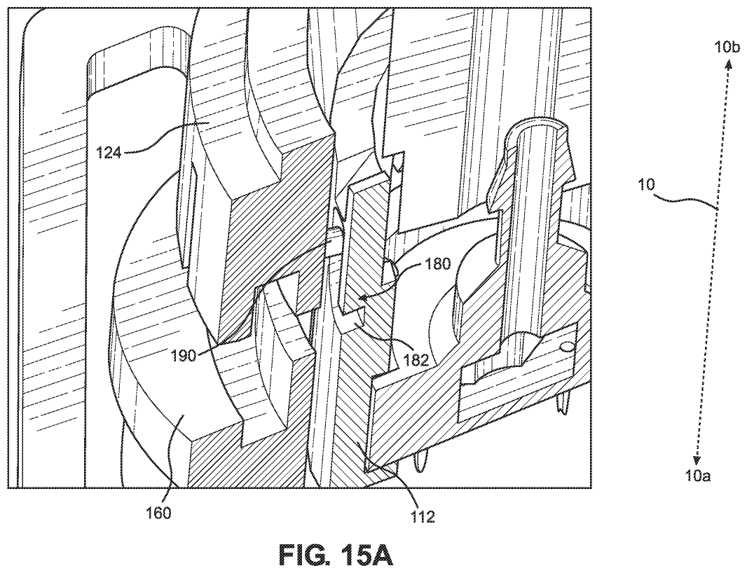

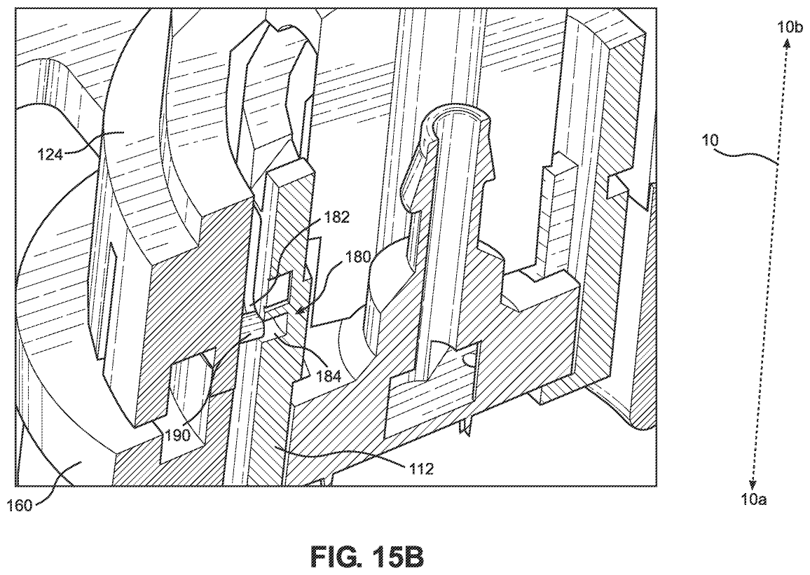

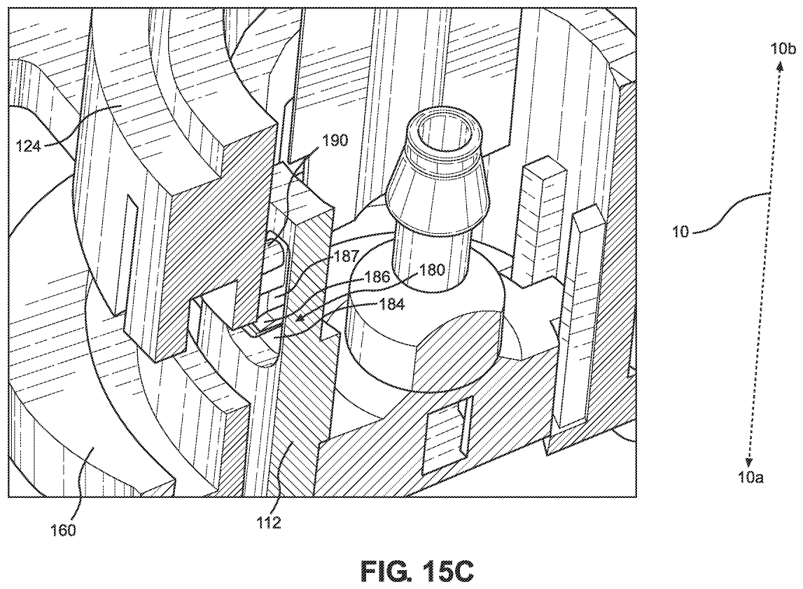

Referring to FIGS. 15A-15C, a mechanism is described that is configured to prevent repeated movement of the needle guard 160 to its retracted configuration. As described previously, the needle guard 160 can be moved from its extended configuration to its retracted configuration when the retaining clips 114 are deflected by movement of the actuation hub 124 relative to the needle hub 112. The exemplary mechanism can include an engagement between the actuation hub 124 and the needle hub 112 that prevents subsequent axial movement of the actuation hub 124 to deflect the retaining clips 114 after the first instance of doing so. A locking track 180 can be defined on the needle hub 112 and configured to slidably receive therein a protrusion 190 extending from the actuation hub 124. In some aspects, the track 180 can be J-shaped, U-shaped, O-shaped, or another suitable shape. The track 180 can be dimensioned such that the protrusion 190 can be received therein and travel along the track 180 during movement of the actuation hub 124 relative to the needle hub 112, or vice versa.

The track 180 can include a first portion 182 (see FIG. 15A), a second portion 184 adjacent to and connected to the first portion 182, and a third portion 187 adjacent to and connected to the second portion 184 (see FIG. 15C). Prior to use, the protrusion 190 of the actuation hub 124 can be positioned in the first portion 182. In some aspects, the protrusion 190 can be positioned outside of the track 180 prior to assembly of the injection device 100.

During use, when the actuation hub 124 is moved in the injection direction 10a, the protrusion 190 on the actuation hub 124 can be slidably moved within the first portion 182 of the track 180. The protrusion 190 can follow the shape of the track 180 as the protrusion 190 moves through the first portion 182 toward the second portion 184. The track 180 can be curved along the first portion 182 and/or the second portion 184. As the protrusion 190 follows the curvature of the track 180, the actuation hub 124 can be rotated about the injection axis 10 in the direction that the protrusion 190 moves as it follows the track 180 (see FIG. 15B). The needle hub 112 can be rotationally fixed with respect to the injection axis 10. It should be appreciated that, in some aspects, the needle hub 112 can be configured to rotate about the injection axis 10 while the actuation hub 124 remains rotationally stationary instead.

When the actuation hub 124 has been moved in the injection direction 10a by a predetermined distance to where the actuation hub 124 has deflected the retaining clips 114 and caused movement of the needle guard 160 to its retraced position, the protrusion 190 moves into the second portion 184 of the track 180, and the actuation hub 124 has rotated about the injection axis 10. When the injection is completed, the coil spring 168 can be configured to cause movement of the actuation hub 124 in the retraction direction 10b. As the actuation hub 124 is moved in the retraction direction 10b, the protrusion 190 is positioned in, and is configured to follow, the second portion 184 of the track 180. The protrusion 190 does not enter the first portion 182 again when the actuation hub 124 moves in the retraction direction 10b.

As the protrusion 190 follows the second portion 184 in the retraction direction 10b, the protrusion 190 can contact a deflectable locking member 186 as shown in FIG. 15C. The deflectable locking member 186 can separate the second portion 184 from the third portion 187 and be configured to obstruct or prevent movement of the protrusion 190 therethrough. The deflectable locking member 186 can be configured to be deflected by the protrusion 190. In some aspects, the deflectable locking member 186 may be deflectable in the retraction direction 10b but not in the injection direction 10a. The deflectable locking member 186 can be biased to its undeflected configuration, such that when the protrusion 190 passes by the deflectable locking member 186, the deflectable locking member 186 returns to its undeflected position to obstruct or block the second portion 184 of the track 180. As the actuation hub 124 is moved in the retraction direction 10b, the protrusion 190 contacts and deflects the deflectable locking member 186 into the third portion 187. The protrusion 190 can then be moved past the deflectable locking member 186 into the third portion 187 of the track 180.

The deflectable locking member 186 can be configured to not be easily deflected in the injection direction 10a. As such, movement of the actuation hub 124 in the injection direction 10a again would be precluded by the contact of the protrusion 190 and the deflectable locking member 186. When the protrusion 190 is in the third portion 187 and the deflectable locking member 186 is in its undeflected position, the protrusion 190 can no longer be move out of the third portion 187 back to the second portion 184.

When the protrusion 190 is blocked from moving in into the first or second portions 182 or 182 in the injection direction 10a, the actuation hub 124 is also precluded from moving in the same direction. If the actuation hub 124 cannot move to the predetermined distance in the injection direction 10a, the actuation hub 124 cannot contact the retaining clips 114 and cause the retaining clips 114 to deflect and allow the needle guard 160 to move into its retracted position. As such, when the protrusion 190 is in the third portion 187, the injection device 100 can not be actuated to pierce the user's skin or commence injection. The injection device 100 can be single-use and not be reused, thus avoiding some of the disadvantages of reusable devices listed above and generally known.

It should be appreciated that although the exemplary embodiment in FIGS. 15A-15C depicts the track 180 on the needle hub 112 and the protrusion 190 on the actuation hub 124, the placement of these components can be reversed, such that the track 180 is on the actuation hub 124 and the protrusion 190 is on the needle hub 112.

Utilizing an ortho-planar spring such as described throughout this application can provide certain advantages over other biasing mechanisms. An ortho-planar spring can be injection-molded out of a suitable injection molding plastic material. The ortho-planar spring can be molded as a unitary part, thus simplifying the manufacturing process and decreasing risk of multi-component failure. Injection molding allows for precise adjustment of the ortho-planar spring to have the physical parameters that are beneficial to specific injection device applications. For example, the size, shape, thickness, quantity, and orientation of the flexible arms 150 can be easily changed when forming an ortho-planar spring via injection molding. Adjustments can be made to result in a desired velocity of injection of medicament. These calculations can be made due to known material properties of the injection molding material to result in the desired elastic compression force needed to move the platform of the ortho-planar spring from the undeflected configuration to the deflected configuration and to ensure a desired potential energy held in the ortho-planar spring in the deflected configuration. Using this potential energy, the material properties of the ortho-planar spring, and the known distance between the needle and the injection surface, manufacturers can calculate the velocity with which the needle is moved towards and into the injection surface. Similarly, adjustments can be made to the ortho-planar spring to control for the desired depth of penetration of the needle into the skin.

An ortho-planar spring as described herein offers a smaller component footprint (at least along the injection axis 10) than some alternative options, which may be beneficial when designing a smaller injection device that would be easier to secure to the user's skin and easier to conceal under clothing. Because portions of the ortho-planar spring (such as the platform, the flexible arms, and the base) can, at different stages of use, be disposed within the same plane, the ortho-planar spring is more compact than other alternatives and can be used in smaller devices.

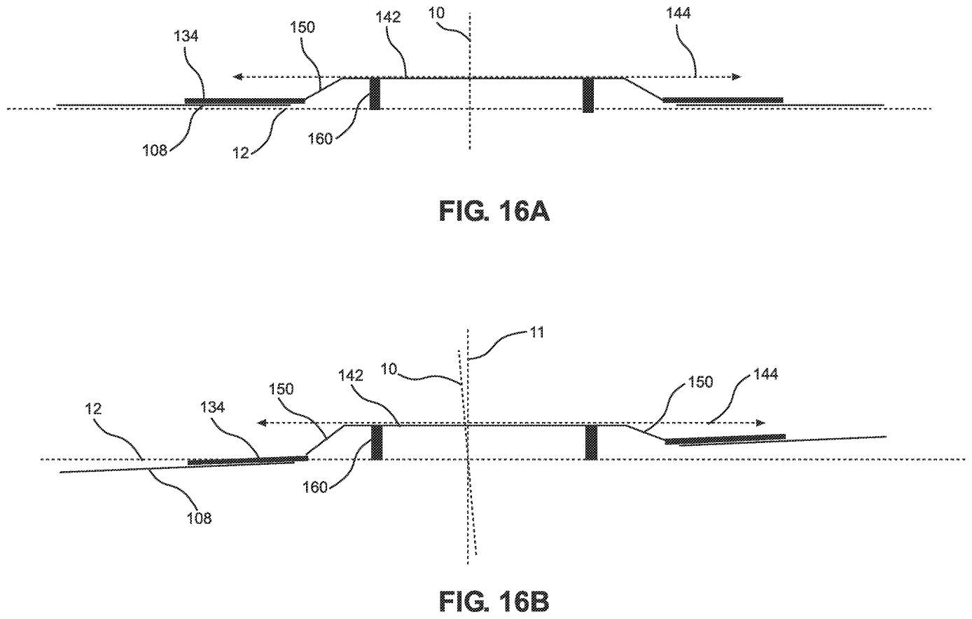

In some aspects, an ortho-planar spring as described herein can be beneficial in improving contact of the injection device 100 with the injection surface on the user's skin. During use, the platform 142 can be moved in the injection direction 10a or in the retraction direction 10b along the injection axis 10. Referring to FIGS. 16A and 16B, in some aspects, the injection surface on the user's skin may be non-parallel to the housing surface 108. The injection plane 12 may be non-parallel to the base plane 154. In such cases, when the injection device 100 contacts the skin, the flexible arms 150 can still be configured to be deflected and moved from the undeflected configuration to the deflected configuration resulting in the ortho-planar spring 130 being moved from the resting configuration to the compressed configuration. As shown in FIG. 16B, if the skin surface may be non-parallel to the surface 108 of the housing 104, and the injection plane 12 is not parallel to the surface 108 or the base plane 154, one or more flexible arms 150 can be deflected appropriately such that the ortho-planar spring 130 still contacts the injection surface and the needle 116 is correctly oriented relative to the skin. It will be appreciated that in such scenarios, one or more of the flexible arms 150 can be deflected to a greater extent than one or more of the other flexible arms 150 based on the forces of the injection surface acting on the platform 142 of the ortho-planar spring 130. During such use, the platform 142 can still be moved in a substantially orthogonal direction to the injection plane 12, even if this orthogonal direction is not orthogonal to the surface 108 of the housing 104. The platform 142 can be moved toward the retraction direction 10b along a secondary axis 11 that can be offset from the injection axis 10. In such instances, when the ortho-planar spring 130 is in its deflected configuration, the platform 142 may lie in a plane that intersects, but is not parallel to, the surface 108. In such aspects, the needle plane 144 may be non-parallel to the base plane 154. The flexible arms 150 may be deflected to different degrees such that, when the ortho-planar spring is in the deflected configuration, the needle 116 affixed to the platform 142 is orthogonal to the user's skin even if the surface 108 of the housing 104 is not orthogonal to the skin. As shown in FIG. 16A, when the injection plane 12 is substantially parallel to the surface 108, the ortho-planar spring 130 can be moved to its deflected configuration such that the arms 150 are deflected substantially evenly. Alternatively, as shown in FIG. 16B, if the injection plane 12 is not parallel to the surface 108, at least one of the flexible arms 150 can be deflected more (for example toward the retraction direction 10b) than one or more of the other flexible arms 150 to result in the platform 142 being substantially parallel to the injection plane 12, such that the needle 116 is substantially orthogonal to the injection plane 12.

Embodiments of injection devices described throughout this application can be incorporated into various drug delivery devices used in the medical field to delivery one or more medicaments to a user. Such devices can include wearable injection devices configured to be temporarily affixed directly to the user's skin for the duration of drug delivery. In some aspects, such devices can include self injectors, auto injectors, and/or other hand-held injectors configured to be operable with one hand by the user. Drug delivery devices can include the medicament therein or, alternatively, can be operationally connected to a medicament source separate from the device. In some aspects, a single drug delivery device can include two or more injection devices as described in this application, for example, to deliver two or more different medicaments to the user, to deliver medicaments to two or more different locations on the user's skin, and/or to deliver medicaments at two or more different delivery profiles (for example, at different flow rates, volumes, delivery conditions, time, sequence, delivery pattern, etc.). Nothing in this application should be construed as limiting the potential application or incorporation of the described injection devices.

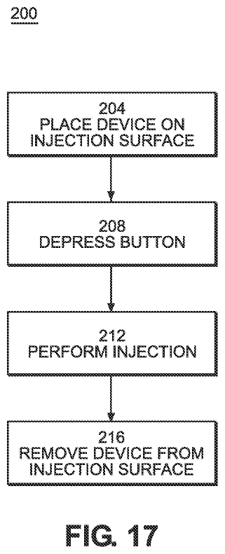

Referring to FIG. 17, an exemplary process 200 is shown that describes steps of using an injection device 100 as described throughout this application. It should be appreciated that the process 200 is not limited to any particular embodiment of the injection device 100 described herein and could apply to any or all embodiments.

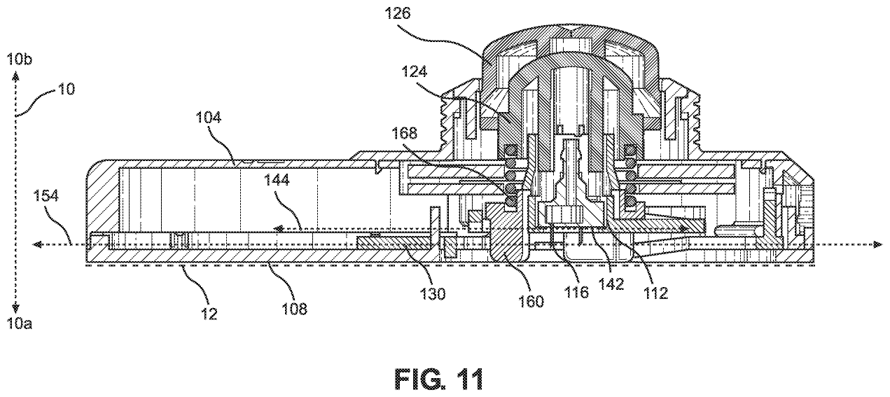

At step 204, a user can place the injection device 100 onto the surface of the skin at the injection site where the medicament will be injected. The injection site can be substantially parallel to, lie in, or define the injection plane 12 described above. Prior to the injection device 100 contacting the skin, the ortho-planar spring 130 can be in its undeflected position as described above and extend out of the housing 104 beyond the surface 108 in the injection direction 10a (see FIG. 10). When the injection device 100 is placed on the skin, the user can apply sufficient force in the injection direction 10a to cause the ortho-planar spring 130 to deflect in the retraction direction 10b such that the platform 142 is moved toward the surface 108. Any components that are fixed to the platform 142 can also be moved in the same direction. In some aspects, the needle hub 112 containing the needle 116 can be moved along with the platform 142. The needle guard 160 may be fixed to the needle hub 112 and may also be moved with the platform 142. The platform 142 can be moved in the retraction direction 10b until the ortho-planar spring 130 and any component attached thereto are no longer between the surface 108 and the injection site on the skin. This allows the surface 108 to contact the injection site in a substantially planar manner. In some aspects, the needle guard 160 can be affixed to the needle hub 112 such that at least a portion of the needle guard 160 extends past the needle tip 118 of the needle 116 in the injection direction 10a, thus blocking the needle tip 118 to prevent the user from accidentally contacting the needle tip 118. In such aspects, when the injection device 100 is placed on the skin and the surface 108 contacts the skin, the needle guard 160 can also contact the skin and be biased in the injection direction 10a by the biasing force of the deflected ortho-planar spring 130 (see FIG. 11).

At step 208, after the injection device 100 is placed on the skin, the user can actuate insertion of the needle 116 into the injection site at the skin. The user can depress the button 126, for example in the injection direction 10a. The button 126 can be pushed until it contacts the actuation hub 124 and moves the actuation hub 124 in the injection direction 10a the preset distance. The actuation hub 124 can be moved until it contacts the retaining clips 114 and deflects the retaining clips 114 as described above to disengage the needle hub 112 from the needle guard 160 along the injection axis 10. Once the needle guard 160 is disengaged from the needle hub 112 (and thus from the platform 142 of the ortho-planar spring 130), the bias of the ortho-planar spring 130 causes the platform 142 to be moved in the injection direction 10a toward the injection site. The needle hub 112 and the needle 116, which are affixed to the platform 142, are also moved in this direction until the needle tip 118 contacts and penetrates the skin at the injection site. The needle 116 may extend past the platform 142 in the injection direction 10a, such that when the platform 142 is moved toward the skin, the needle tip 118 contacts the skin before the platform 142 does. The needle guard 160 can be in its retracted configuration at this stage, such that the needle guard 160 does not axially extend past the needle tip 118 in the injection direction 10a. As such, the needle tip 118 can contact the skin and not be blocked off by the needle guard 160.

In some aspects, the actuation of insertion of the needle 116 into the injection site can be triggered by the action of placing the injection device 100 onto the injection surface during step 204. In such aspects, the deflection of the ortho-planar spring 130 in the retraction direction 10b can cause the disengagement of the needle hub 112 from the needle guard 160 and allow the needle hub 112 to be moved along the injection axis 10 as described above. In such arrangements, step 208 can occur automatically as step 204 occurs without additional action from the user.

The needle 116 can be driven into the skin by the force of the ortho-planar spring 130 (see FIG. 13). The depth of the needle 116 penetrating the skin can be controlled by selecting the length or thickness of the needle 116, the force of the ortho-planar spring 130 acting on the needle hub 112, the distance between the needle tip 118 and the platform 142, and/or other parameters. In some aspects, the needle 116 is moved into the skin until the platform 142 contacts the skin.

After the needle 116 is moved into the skin, the medicament can be injected at step 212. The injection can be accomplished by a drive system 28 configured to move the medicament from the medicament source 20 through the needle 116 and into the skin at the injection site. In some aspects, the injection device 100 may be configured to actuate the drive system 28 automatically after step 208 has been performed and the needle 116 was moved into the skin. In some aspects, the user can separately actuate the drive system 28 to cause the medicament to be injected.

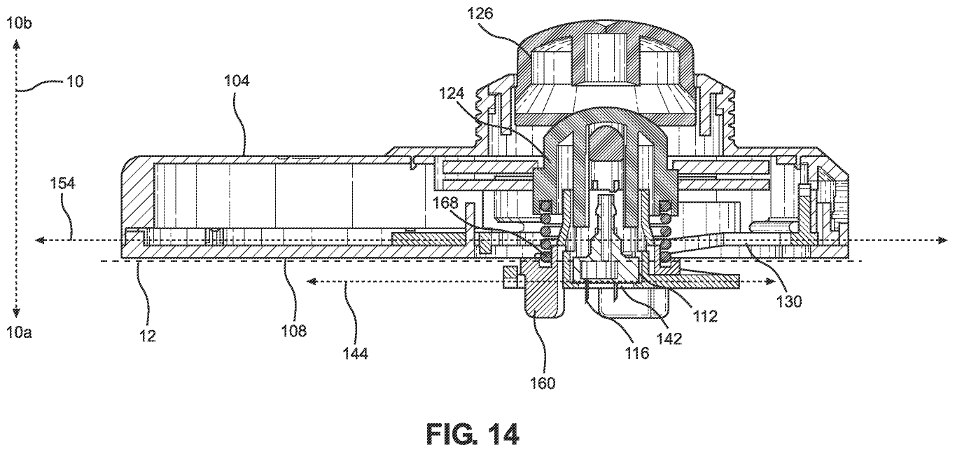

At step 216, the user can remove the injection device 100 from the injection site. This can be done after injection of the medicament has been completed. As the injection device 100 is moved and the surface 108 is separated from the skin, the ortho-planar spring 130, which has been deflected and is biased in the injection direction 10a, can revert to its undeflected configuration. The deflected flexible arms 150 can cause the platform 142 to move in the injection direction 10a back to its original position at rest, and any components affixed to the platform 142 can be moved as well. Prior to step 216, the needle guard 160 is in its retracted configuration and contacts the skin while the injection device 100 is placed on the skin. As such, the needle guard 160 is precluded from being moved in the injection direction 10a while the injection device 100 is on the skin. When the injection device is moved away from the skin, the needle guard 160 is no longer precluded from moving in the injection direction 10a. In some aspects, the coil spring 168 can apply a biasing force onto the needle guard 160 in the injection direction 10a to push the needle guard 160 in the same direction. The needle guard 160 can be moved in the injection direction 10a until the needle guard 160 passes the distal end 117 of each retaining clip 114. Once the needle guard 160 has passed the distal end 117, the ramp portion 115 of each retaining clip 114 can radially move back to their respective undeflected configurations, thus positioning the distal ends 117 in axial alignment with the needle guard 160 along the injection axis 10. At this point, the needle guard 160 is in its extended configuration and extends beyond the needle tip 118 in the injection direction 10a again (see FIG. 14).

In some aspects, the actuation hub 124 can be moved in the retraction direction 10b when the injection device 100 is removed from the skin. Movement of the actuation hub 124 can be caused by the coiled spring 168 that can apply a biasing force on the actuation hub 124 in the retraction direction 10b. As the actuation hub 124 is moved in the retraction direction 10b, the protrusion 190 can follow the second portion of the locking track 180 and move into the third portion 187 by passing the deflectable locking member 186 as described above. The actuation hub 124 can be rotated as the protrusion 190 follows the track 180 at one or more steps between, and including, steps 208 and 216. After step 216 is performed, the injection device 100 may be configured to not be reusable to move the needle 116 into the user and/or to deliver medicament again. When the protrusion 190 is in the third portion 187 of the locking track 180 and the deflectable locking member 186 is in its undeflected position, the protrusion 190 cannot be moved out of the third portion 187. The actuation hub 124, to which the protrusion 190 is fixedly attached, thus cannot be moved in the injection direction 10a to contact and deflect the retaining clips 114. As such, the needle guard 160 can no longer be moved to its retracted configuration to expose the needle tip 118.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined in methods consistent with various embodiments of the present invention. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence. The methods described herein can include additional steps performed before the first step, after the last step, or anywhere else in the process.

When values are expressed as approximations by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, reference to values stated in ranges includes each and every value within that range.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Features of the disclosure that are described above in the context of separate embodiments may be provided in combination in a single embodiment. Conversely, various features of the disclosure that are described in the context of a single embodiment may also be provided separately or in any sub-combination. Finally, while an embodiment may be described as part of a series of steps or part of a more general structure, each said step may also be considered an independent embodiment in itself, combinable with others.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

What is claimed:

1. An injection device for delivering a medicament to an injection site, the injection device comprising:
a housing configured to be placed on the injection site, the housing defining a contact surface thereon configured to contact the injection site;
an ortho-planar spring disposed in the housing and configured to be moved between a resting configuration and a compressed configuration;
a needle hub fixedly attached to the ortho-planar spring;
a needle affixed to the needle hub, the needle having a needle tip at one end thereof;
a needle guard having an extended configuration, in which the needle guard covers the needle tip, and a retracted configuration, in which the needle guard does not cover the needle tip; and
a retaining clip configured to contact the needle guard and to prevent axial movement of the needle guard from the extended configuration to the retracted configuration,
wherein the ortho-planar spring is configured to be moved to the compressed configuration when the housing is placed on the injection site such that the needle hub and the needle guard are moved axially toward the housing, and
wherein the retaining clip is configured to be deflected away from the needle guard to allow relative movement between the needle guard and the needle hub such that the needle guard is transitioned to the retracted configuration.

2. The injection device of claim 1, wherein the ortho-planar spring includes a base affixed to the housing, a platform configured to be moved axially along an injection axis, and a plurality of flexible arms connecting the base to the platform, wherein the needle hub is fixedly attached to the platform.

3. The injection device of claim 1, wherein when the ortho-planar spring is in the resting configuration, the platform is axially spaced away from the base along the injection axis.

4. The injection device of claim 3, wherein when the injection device is placed on the injection site, the platform of the ortho-planar spring is moved axially toward the base to place the ortho-planar spring into the compressed configuration, the ortho-planar spring being biased to its resting configuration.

5. The injection device of claim 1, further comprising a spring configured to bias the needle guard to the extended configuration.

6. The injection device of claim 1, further comprising an actuation hub that is axially movable along the injection axis and is configured to contact the retaining clip to cause the retaining clip to deflect away from the needle guard.

7. The injection device of claim 1, further comprising a locking mechanism configured to prevent reuse of the injection device, the locking mechanism comprising:
a protrusion on one of the actuation hub or the needle hub and a guide track on the other of the actuation hub or the needle hub, wherein the protrusion is configured to follow the guide track from a first position to a second position and precluded from following the guide track from the second position to the first position.

8. The injection device of claim 7, wherein the protrusion is disposed on the actuation hub, and the guide track is defined on the needle hub.

9. The injection device of claim 7, wherein the guide track is curved, and at least one of the actuation hub or the needle hub is configured to rotate about the injection axis as the protrusion follows the curved guide track.

10. The injection device of claim 1, further comprising a button disposed on the housing and configured to be depressed to cause the retaining clip to be deflected away from the needle guard.

11. The injection device of claim 1, wherein the contact surface of the housing includes an adhesive.

12. The injection device of claim 1, wherein the needle is a microneedle.

13. The injection device of claim 1, wherein the injection device comprises a plurality of needles.

14. The injection device of claim 1, wherein the injection device comprises a plurality of retaining clips.

15. The injection device of claim 1, further comprising a medicament source configured to retain the medicament therein, the medicament source being in fluid communication with the needle.

16. The injection device of claim 1, wherein the needle guard is axially fixed relative to the needle hub when the needle guard is in the extended configuration.

17. The injection device of claim 1, further comprising:
a medicament; and
an actuation mechanism configured to move the medicament through the injection device and to a user.

18. The injection device of claim 1, wherein the injection device is a wearable injection device configured to be attached to the user.

19. The injection device of claim 17, wherein the injection device is a hand-held injector.

20. A needle safety system for use with an injection device, the needle safety system comprising:
an ortho-planar spring having a base and a platform separated from the base by a plurality of flexible arms;
a needle hub attached to the platform and including a needle thereon, the needle having a needle tip at one end thereof;
a needle guard having an extended configuration, in which the needle guard covers the needle tip, and a retracted configuration, in which the needle guard does not cover the needle tip; and
a retaining clip extending from the needle hub and configured to contact the needle guard, the retaining clip having an undeflected configuration and a deflected configuration,
wherein when the retaining clip is in the undeflected configuration, the retaining clip contacts the needle guard and prevents relative axial movement between the needle guard and the needle hub, and when the retaining clip is in the deflected configuration, relative movement between the needle guard and the needle hub is permitted.

21-32. (canceled)
